(19) United States
(12) Patent Application Publication
(10) Pub. No.: US 2024/0342160 A1
(43) Pub. Date: Oct. 17, 2024

(54) VALBENAZINE FOR USE IN THE TREATMENT OF DYSKINESIA DUE TO CEREBRAL PALSY

(71) Applicant: Neurocrine Biosciences, Inc., San Diego, CA (US)

(72) Inventor: Grace S. Liang, San Diego, CA (US)

(21) Appl. No.: 18/575,126

(22) PCT Filed: Jun. 28, 2022
(86) PCT No.: PCT/US2022/073214

§ 371 Date: Dec. 28, 2023

Related U.S. Application Data
(60) Provisional application No. 63/298,291, filed on Jan. 11, 2022.
(60) Provisional application No. 63/279,815, filed on Nov. 16, 2021.
(60) Provisional application No. 63/216,877, filed on Jun. 30, 2021.

(51) Int. Cl. A61K 31/4745

(52) U.S. Cl. CPC A61K 31/4745

(57) ABSTRACT

Provided is a method of treating dyskinesia due to cerebral palsy in a patient in need thereof, comprising: administering a vesicular monoamine transporter 2 (VMAT2) inhibitor to the patient in need thereof.

Cerebral palsy (CP) is a neurodevelopmental disorder that begins in early childhood and is persistent throughout life, affecting about 3 in 1000 children in the US. Globally, the prevalence of CP is 1.7 to 3.1 per 1000 live births in high-income countries, with a higher prevalence in low-income countries. CP may result from perinatal hypoxic-ischemic injury, stroke, infection, metabolic disturbance (i.e., kernicterus), or other mechanisms that affect development of the basal ganglia and other brain structures involved in motor control. CP is clinically categorized into spastic, dyskinetic, and ataxic subtypes based on the predominant motor disorder.

People with dyskinetic cerebral palsy (DCP, also known as athetoid cerebral palsy or ADCP) is a form of cerebral palsy marked by involuntary movement, resulting in an overall severe motor impairment. There are three defining characteristics of dyskinetic cerebral palsy: dystonia, athetosis, and chorea. DCP accounts for up to 15% of CP cases globally.

Patients with DCP often have mixed phenotypes with varying degrees of dystonia and choreoathetosis, and the approaches for treating these motor manifestations are different. Currently, no therapies are approved for the treatment of dystonic or choreoathetoid forms of DCP. The most common oral pharmacological therapies used in clinical practice for the treatment of dystonia in DCP include anticholinergics (trihexyphenidyl and benztropine), the gamma-aminobutyric acid (GABA)-B receptor agonist baclofen, and benzodiazepines (diazepam and clonazepam). Intramuscular botulinum toxin injections are also used for focal treatment of dystonia. Antiepileptics, benzodiazepines, and anticholinergics are also used as treatments for choreoathetosis with varying levels of evidence for efficacy and well-known potential for adverse effects. In more severe cases, surgical approaches such as deep brain stimulation (DBS) have also been tried. Pharmacological treatments for the dyskinetic/choreoathetoid forms of DCP may improve one symptom and worsen another, or induce adverse effects before reaching the therapeutic dose. Effectively treating the range of disabling motor symptoms experienced by patients with DCP while minimizing off-target effects remains an important unmet medical need.

There is a significant, unmet need for methods for treating cerebral palsy, such as dyskinetic cerebral palsy. The present disclosure fulfills these and other needs, as evident in reference to the following disclosure.

### SUMMARY

Provided is a method of treating dyskinesia due to cerebral palsy in a patient in need thereof, comprising administering to the patient a therapeutically effective amount of valbenazine, or an isotopic variant thereof, or a pharmaceutically acceptable salt of valbenazine or an isotopic variant thereof. In some embodiments, the valbenazine, or an isotopic variant thereof, or a pharmaceutically acceptable salt of valbenazine or an isotopic variant thereof, is administered via a titration scheme that comprises the up-titration of the valbenazine, or an isotopic variant thereof, or a pharmaceutically acceptable salt of valbenazine or an isotopic variant thereof, over a period of no more than about six weeks until an optimized dose is administered.

These and other aspects of the invention will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF THE FIGURES

FIG. 1 shows a schematic of the study design. Randomization will be stratified by age group (6 to 11 years, 12 to 17 years, and 18 to 70 years) and stable concomitant botulinum toxin regimen (yes/no) at baseline (Day 1). Valbenazine dose for pediatric patients <50 kg starts at 20 mg/day. Valbenazine dose for pediatric patients ≥50 kg and adults starts at 40 mg/day. For patients who do not tolerate a dose increase, one dose level reduction is allowed from the highest titrated dose during each treatment period. A follow-up visit will occur 2 weeks after the last dose of study treatment. Patients who discontinue study treatment early during the double-blind treatment period should remain in the study to complete Week 14 study assessments. PBO=placebo; R=randomization; VBZ=valbenazine.

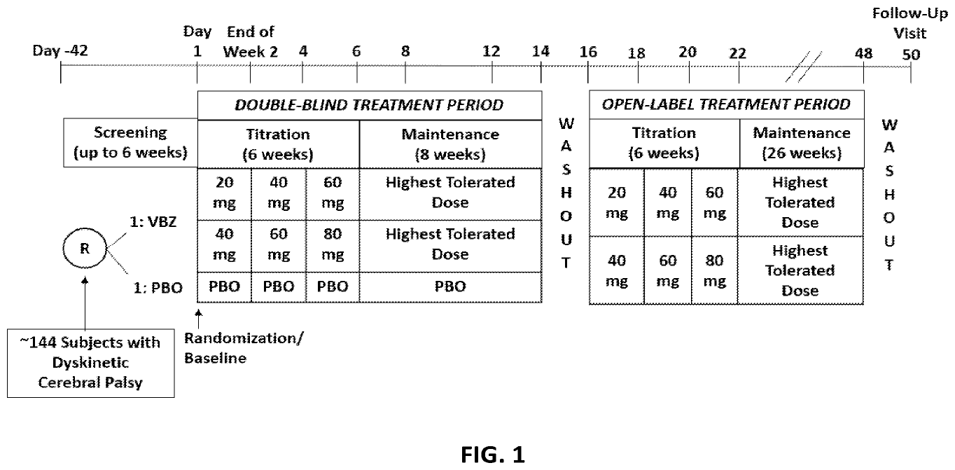

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, in some embodiments, “pharmaceutically acceptable salt” refers to acid addition salts with an inorganic or an organic acid. Lists of suitable salts are found in WO 87/05297, Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977), each of which is incorporated herein by reference in its entirety. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002 which is incorporated herein by reference in its entirety. The organic or inorganic acids include, but are not limited to, hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and the like. In some embodiments, “pharmaceutically acceptable salt” refers to base addition salts with an inorganic or an organic base. Inorganic bases which may be used to prepare salts include, for example, sodium, potassium, lithium, ammonium, calcium, magnesium, iron, zinc, manganese, aluminum hydroxides, carbonates, bicarbonates, phosphates, and the like; particularly preferred are the ammonium, potassium, sodium, calcium, and magnesium hydroxides, carbonates, bicarbonates, or phosphates. Organic bases from which may be used to prepare salts include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

As used herein, “about” mean±20% of the stated value, and includes more specifically values of ±10%, +5%, +2% and +1% of the stated value.

As used herein, “baseline” refers to the period of time just prior to initiation of therapy. The patient's condition just prior to initiation of therapy can be referred to as the patient's baseline condition.

As used herein, “adjusting administration”, “altering administration”, “adjusting dosing”, or “altering dosing” are all equivalent and mean tapering off, reducing or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein, “athetosis” refers to another characteristic of dyskinetic cerebral palsy, marked by slow twitching and wriggling movements. The symptoms can surface while resting and generally become worse when the individual moves. Other common symptoms of athetosis include: Involuntary slow, continuous writhing movements which worsen with attempts to move; fluctuating muscle tone (from stiff to floppy); grimacing and/or drooling from lack of facial muscle control; difficulties with eating and drinking; difficulties grasping and holding small objects because of changes in muscle tone; involuntary movements may be continuous unless the individual is totally relaxed; and involuntary movements typically disappear when the individual is asleep.

As used herein, “chorea” refers to a characteristic of dyskinetic cerebral palsy marked by brief irregular and involuntary movements (irregular migrating contractions). The name was derived from the Greek word, chorea, meaning “dance,” since the involuntary movements are often repetitive. This can affect multiple parts of the body. Chorea can result in difficulties with chewing, drinking, swallowing, and speech.

As used herein, “choreoathetosis” or “choreoathetoid” is the occurrence of involuntary movements in a combination of chorea (irregular migrating contractions) and athetosis (twisting and writhing).

As used herein, “dystonia” refers to involuntary muscle contractions that are marked by writhing, slow and repetitive movements that become worse when the individual begins to move. Other symptoms include abnormal and awkward posture, movements that alternate from slow and painful to fast and rapid, and involuntary movements that increase when the child is stressed or tired. Dystonia can affect all parts or the body or be localized in only one area of the body.

As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, a “dose” means the measured quantity of an active agent to be taken at one time by a patient. In certain embodiments, wherein the active agent is not valbenazine free base, the quantity is the molar equivalent to the corresponding amount of valbenazine free base. For example, often a drug is packaged in a pharmaceutically acceptable salt form, for example valbenazine ditosylate, and the dosage for strength refers to the mass of the molar equivalent of the corresponding free base, valbenazine. As an example, 73 mg of valbenazine tosylate is the molar equivalent of 40 mg of valbenazine free base.

As used herein, “dosing regimen” means the dose of an active agent taken at a first time by a patient and the interval (time or symptomatic) at which any subsequent doses of the active agent are taken by the patient such as from about 20 to about 160 mg once daily, e.g., about 20, about 40, about 60, about 80, about 100, about 120, or about 160 mg once daily. The additional doses of the active agent can be different from the dose taken at the first time.

As used herein, a “dosage” is the prescribed administration of a specific amount, number, and frequency of doses over a specific period of time.

As used herein, “effective amount” and “therapeutically effective amount” of an agent, compound, drug, composition, or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a compound contains an unnatural proportion of deuterium.

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position. The isotopic enrichment of the compounds provided herein can be determined using conventional analytical methods known to one of ordinary skill in the art, including mass spectrometry, nuclear magnetic resonance spectroscopy, and crystallography.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “patient” or “individual” or “subject” means a mammal, including a human, for whom or which therapy is desired, and generally refers to the recipient of the therapy.

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, the terms “point” and “score” are used interchangeably herein, and refer to the measure of a certain rating scale.

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “up-titration” of a compound refers to increasing the amount of a compound to achieve a therapeutic effect that occurs before dose-limiting intolerability for the patient. Up-titration can be achieved in one or more dose increments, which may be the same or different.

As used herein, “valbenazine” may be referred to as(S)-2-amino-3-methyl-butyric acid (2R, 3R,11bR)-3-isobutyl-9,10-dimethoxy-1,3,4,6,7,11b-hexahydro-2H-pyrido[2,1-a]isoquinolin-2-yl ester; or as L-Valine, (2R,3R,11bR)-1,3,4,6,7,11b-hexahydro-9,10-dimethoxy-3-(2-methylpropyl)-2H-benzo[a]quinolizin-2-yl ester or as NBI-98854 and has the following chemical structure:

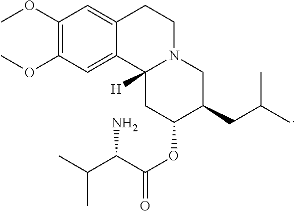 [SMILES: COc1cc2c(cc1OC)[C@H]1C[C@@H](OC(=O)[C@@H](N)C(C)C)[C@H](CC(C)C)CN1CC2]

Valbenazine can be prepared according to U.S. Pat. Nos. 8,039,627 and 8,357,697, the disclosure of each of which is incorporated herein by reference in its entirety. In another embodiment, the valbenazine for use in the compositions and methods provided herein is in polymorphic Form I as disclosed in U.S. Ser. No. 15/338,214, the disclosure of which is incorporated herein by reference in its entirety.

As used herein, “VMAT2” refers to human vesicular monoamine transporter isoform 2, an integral membrane protein that acts to transport monoamines, particularly neurotransmitters such as dopamine, norepinephrine, serotonin, and histamine, from cellular cytosol into synaptic vesicles.

As used herein, the term “VMAT2 inhibitor”, “inhibit VMAT2”, or “inhibition of VMAT2” refers to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The term “VMAT2 inhibitor”, “inhibit VMAT2”, or “inhibition of VMAT2” also refers to altering the function of VMAT2 by decreasing the probability that a complex forms between a VMAT2 and a natural substrate.

Provided is a method of treating dyskinesia due to cerebral palsy in a patient in need thereof, comprising administering to the patient a therapeutically effective amount of vesicular monoamine transport 2 (VMAT2) inhibitor. Also provided is a method of treating dyskinesia due to cerebral palsy in a patient in need thereof, comprising administering to the patient a therapeutically effective amount of vesicular monoamine transport 2 (VMAT2) inhibitor selected from valbenazine, or a pharmaceutically acceptable salt thereof, or an isotopic variant of valbenazine or a pharmaceutically acceptable salt thereof, wherein the VMAT2 inhibitor is administered via a titration scheme that comprises the up-titration of the VMAT2 inhibitor over a period of no more than about six weeks until an optimized dose is administered.

In some embodiments, the dyskinesia due to cerebral palsy is characterized by abnormal involuntary movements of the dystonic type.

In some embodiments, the dyskinesia due to cerebral palsy is characterized by abnormal involuntary movements of the athetoid type.

In some embodiments, the dyskinesia due to cerebral palsy is characterized by abnormal involuntary movements of the chorea type.

In some embodiments, the titration scheme comprises administering the VMAT2 inhibitor at an initial dose equivalent to about 20 mg of valbenazine free base once daily for about two weeks for pediatric subjects having a body weight less than 50 kg and, provided that the patient tolerates the initial dose and that the patient has not achieved satisfactory control of abnormal involuntary movements, increasing the dose and administering the increased dose to the patient. In some embodiments, the increased dose is equivalent to about 40 mg of valbenazine free base once daily. In some embodiments, the titration scheme further comprises administering the VMAT2 inhibitor at said increased dose for about two weeks for pediatric subjects having a body weight less than 50 kg. In some embodiments, if the subject does not tolerate the increased dose, the optimized dose is the initial dose. In some embodiments, if the subject tolerates the increased dose and if the subject has achieved satisfactory control of abnormal involuntary movements, the optimized dose is the increased dose. In some embodiments, the method further comprises administering the optimized dose of the VMAT2 inhibitor to the pediatric subjects having a body weight less than 50 kg. In some embodiments, if the patient tolerates the increased dose and if the patient has not achieved satisfactory control of abnormal involuntary movements, the method further comprises increasing the dose. In some embodiments, the further increased dose is equivalent to about 60 mg of valbenazine free base once daily. In some embodiments, if the patient does not tolerate the further increased dose, the optimized dose is the increased dose. In some embodiments, if the patient tolerates the further increased dose and if the patient has achieved satisfactory control of abnormal involuntary movements, the optimized dose is the further increased dose. In some embodiments, the method further comprises administering the optimized dose of the VMAT2 inhibitor to the patient.

In some embodiments, the titration scheme comprises administering the VMAT2 inhibitor at an initial dose equivalent to about 40 mg of valbenazine free base once daily for about two weeks for patients having a body weight greater than or equal to 50 kg and, provided that the patient tolerates the initial dose and that the patient has not achieved satisfactory control of abnormal involuntary movements, increasing the dose and administering the increased dose to the patient. In some embodiments, the increased dose is equivalent to about 60 mg of valbenazine free base once daily. In some embodiments, the titration scheme further comprises administering the VMAT2 inhibitor at said increased dose for about two weeks to patients having a body weight greater than or equal to 50 kg. In some embodiments, if the patient does not tolerate the increased dose, the optimized dose is the initial dose. In some embodiments, if the patient tolerates the increased dose and patient has achieved satisfactory control of abnormal involuntary movements, the optimized dose is the increased dose. In some embodiments, the method further comprises administering the optimized dose of the VMAT2 inhibitor to the patient. In some embodiments, if the patient tolerates the increased dose and if the patient has not achieved satisfactory control of abnormal involuntary movements, the method further comprises increasing the dose. In some embodiments, the further increased dose is equivalent to about 80 mg of valbenazine free base once daily. In some embodiments, if the patient does not tolerate the further increased dose, the optimized dose is the increased dose. In some embodiments, if the patient tolerates the increased dose and if the patient has achieved satisfactory control of abnormal involuntary movements, the optimized dose is the further increased dose. In some embodiments, the method further comprises administering the optimized dose of the VMAT2 inhibitor to the patients having a body weight greater than or equal to 50 kg.

In some embodiments, the patient is 6 to 11 years. In some embodiments, the patient is 6 to 11 years and weighs <50 kg. In some embodiments, the patient is 6 to 11 years and weighs ≥50 kg.

In some embodiments, the patient is 12 to 17 years. In some embodiments, the patient is 12 to 17 years and weighs <50 kg. In some embodiments, the patient is 12 to 17 years and weighs ≥50 kg.

In some embodiments, the patient is 18 or older. In some embodiments, the patient is 18 or older and weighs <50 kg. In some embodiments, the patient is 18 or older and weighs ≥50 kg.

In some embodiments, prior to administration, the patient has moderate or severe DCP.

In some embodiments, prior to administration, the patient has a Clinical Global Impression of Severity (CGI-S) score of at least 4.

In some embodiments, the patient's abnormal involuntary movements associated with DCP are measured using a clinical assessment instrument, such as, for example, at least one of the rating scales: Unified Huntington Disease Rating Scale-Total Motor Score; Movement Disorders-Childhood Rating Scale Part I; Clinical Global Impression of Severity; and/or Clinical Global Impression of Improvement.

In some embodiments, efficacy will be assessed using a patient or caregiver reported outcome, such as the Patient Global Impression of Improvement, Caregiver Global Impression of Improvement; Cerebral Palsy Quality of Life-Child; Cerebral Palsy Quality of Life-Teen; and/or the Quality of Life in Neurological Disorders.

In some embodiments, the treatment results in a change in the UHDRS TMC score.

In some embodiments, the treatment results in a change in the UHDRS TMD score.

In some embodiments, the treatment results in a change in CGI-S score.

In some embodiments, the treatment results in a change in the UHDRS functional assessment and functional capacity scores.

In some embodiments, the treatment results in a change in MD-CRS Part I score.

In some embodiments, the treatment results in a change in Neuro-QoL.

In some embodiments, the treatment results in a change in CP QOL-Teen.

In some embodiments, the treatment results in a change in CP QOL-Teen (caregiver-proxy report).

In some embodiments, the treatment results in a change in CP QOL-Child.

In some embodiments, the treatment results in a change in CP QOL-Child (caregiver-proxy report).

In some embodiments, the treatment results in a change in the UHDRS TM.

In some embodiments, the treatment results in an improvement in any one or more of the following: UHDRS TMC score; UHDRS TMD score; CGI-S score; UHDRS functional assessment and functional capacity scores; MD-CRS Part I score; Neuro-QoL; CP QOL-Teen; CP QOL-Teen (caregiver-proxy report); CP QOL-Child; and CP QOL-Child (caregiver-proxy report).

In some embodiments, the treatment results in a reduction in the patient's abnormal involuntary movements associated with DCP, relative to the patient's abnormal involuntary movements associated with DCP at baseline.

In some embodiments, the treating results in maintaining the patient's abnormal involuntary movements associated with DCP, relative to the patient's abnormal involuntary movements associated with DCP at baseline. As used in this context, “maintaining the patient's abnormal involuntary movements” means that the patient's abnormal involuntary movements associated with DCP do not change relative to baseline. Thus, in some embodiments, the patient's abnormal involuntary movements associated with DCP remain stabilized, they do not improve but do not worsen.

In some embodiments, the patient's motor function is improved relative to the patient's motor function at baseline following administration of the VMAT2 inhibitor.

In some embodiments, the patient's motor function in the eye and periorbital region is improved relative to the patient's motor function at baseline following administration of the VMAT2 inhibitor.

In some embodiments, the patient's motor function in the face is improved relative to the patient's motor function at baseline following administration of the VMAT2 inhibitor.

In some embodiments, the patient's motor function in the tongue and perioral region is improved relative to the patient's motor function at baseline following administration of the VMAT2 inhibitor.

In some embodiments, the patient's motor function in the neck is improved relative to the patient's motor function at baseline following administration of the VMAT2 inhibitor.

In some embodiments, the patient's motor function in the trunk is improved relative to the patient's motor function at baseline following administration of the VMAT2 inhibitor.

In some embodiments, the patient's motor function in the lower limbs is improved relative to the patient's motor function at baseline following administration of the VMAT2 inhibitor.

In some embodiments, the patient's oral/verbal function is improved relative to the patient's oral/verbal function at baseline following administration of the VMAT2 inhibitor.

In some embodiments, the patient's self-care function is improved relative to the patient's self-care function at baseline following administration of the VMAT2 inhibitor.

In some embodiments, the patient's attention/alertness is improved relative to the patient's attention/alertness at baseline following administration of the VMAT2 inhibitor.

In some embodiments, safety of the treatment is measured by one or more of the following assessments: Columbia-Suicide Severity Rating Scale, the Hospital Anxiety and Depression Scale, the Children's Depression Inventory 2^{nd} Edition, the Barnes Akathisia Rating Scale, The Modified Ashworth Scale, and the UHDRS Items for Parkinsonism.

In some embodiments, the patient is further administered an additional therapeutic agent. As used herein, an additional therapeutic agent refers to an agent other than the VMAT2 inhibitor that is administered to treat an aspect of the patient's cerebral palsy, such as, for example, eye movement abnormalities, communication problems, swallowing difficulty, poor weight gain, social isolation, hip dysplasia and dislocation, scoliosis, osteopenia and fractures, pain, and movement disorders. Exemplary additional therapeutic agents include anticholinergics (e.g., benztropine mesylate, carbidopa-levodopa, glycopyrrolate, procyclidine hydrochloride, and trihexyphenidyl hydrochloride), anticonvulsants (e.g., gabapentin, lamotrigine, oxcarbazepine, topiramate, and zonisamide), antidepressants (e.g., citalopram, escitalopram, fluoxetine, paroxetine, and sertraline), antispastic (e.g., botulinum toxin, diazepam, dantrolene, cyclobenzadrine, intrathecal baclofen, and tizanidine), and anti-inflammatoires (e.g., aspirin, corticosteroids, nonsteroidal anti-inflammatory drugs (NSAIDs), and steroids). In some embodiments, the administration of the additional therapeutic agent in concomitant with, prior to, or following the administration of the compound of Formula (I), Formula (II) or a combination thereof.

In some embodiments, the valbenazine, or an isotopic variant thereof, or a pharmaceutically acceptable salt of valbenazine or an isotopic variant thereof, can be administered for the treatment of dyskinesia due to cerebral palsy, according to the methods disclosed in U.S. Pat. Nos. 10,857,137; 10,874,648; 10,912,771; 10,940,141; 10,952,997; 10,857,148; and 10,993,941, the disclosure of each of which is incorporated herein by reference in its entirety. In some embodiments, the valbenazine, or an isotopic variant thereof, or a pharmaceutically acceptable salt of valbenazine or an isotopic variant thereof, can be administered for the treatment of dyskinesia due to cerebral palsy, according to the methods disclosed in U.S. Ser. Nos. 17/080,343 and 16/870,572, the disclosure of each of which is incorporated herein by reference in its entirety.

In some embodiments, provided herein is method for the treatment of dyskinesia due to cerebral palsy, wherein the patient is also being administered a strong cytochrome P450 2D6 (CYP2D6) inhibitor, comprising:
orally administering once daily to the patient the valbenazine, or an isotopic variant thereof, or a pharmaceutically acceptable salt of valbenazine or an isotopic variant thereof, in an amount equivalent to about 40 mg as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the treatment of dyskinesia due to cerebral palsy, wherein the patient is a cytochrome P450 2D6 (CYP2D6) poor metabolizer, comprising:
orally administering once daily to the patient the valbenazine, or an isotopic variant thereof, or a pharmaceutically acceptable salt of valbenazine or an isotopic variant thereof, in an amount of equivalent to about 40 mg as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the treatment of dyskinesia due to cerebral palsy, comprising:
(a) orally administering to the patient a therapeutically effective amount of the valbenazine, or an isotopic variant thereof, or a pharmaceutically acceptable salt of valbenazine or an isotopic variant thereof;
(b) subsequently determining that the patient is a cytochrome P450 2D6 (CYP2D6) poor metabolizer; and
(c) reducing dosage of the valbenazine, or an isotopic variant thereof, or a pharmaceutically acceptable salt of valbenazine or an isotopic variant thereof, administered to the patient to an amount equivalent to about 40 mg once daily as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the treatment of dyskinesia due to cerebral palsy, comprising:
determining if the patient is a poor metabolizer of cytochrome P450 2D6 (CYP2D6); and
if the patient is a poor metabolizer of cytochrome P450 2D6 (CYP2D6), then orally administering to the patient a first therapeutically effective amount of the valbenazine, or an isotopic variant thereof, or a pharmaceutically acceptable salt of valbenazine or an isotopic variant thereof, wherein the first therapeutically effective amount is an amount equivalent to about 40 mg once daily as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester; or
if the patient is not a poor metabolizer of cytochrome P450 2D6 (CYP2D6), then orally administering to the patient a second therapeutically effective amount of the valbenazine, or an isotopic variant thereof, or a pharmaceutically acceptable salt of valbenazine or an isotopic variant thereof, wherein the second therapeutically effective amount is an amount equivalent to about 40 mg once daily as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester for one week, and subsequently administering an increased amount of the valbenazine, or an isotopic variant thereof, or a pharmaceutically acceptable salt of valbenazine or an isotopic variant thereof, after one week.

In some embodiments, provided herein is a method for the treatment of dyskinesia due to cerebral palsy, comprising:
(a) orally administering to the patient a therapeutically effective amount of the valbenazine, or an isotopic variant thereof, or a pharmaceutically acceptable salt of valbenazine or an isotopic variant thereof, wherein the therapeutically effective amount is an amount equivalent to about 40 mg once daily as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
(b) subsequently determining that the patient is a poor metabolizer of cytochrome P450 2D6 (CYP2D6); and
(c) administering the same therapeutically effective amount of the valbenazine, or an isotopic variant thereof, or a pharmaceutically acceptable salt of valbenazine or an isotopic variant thereof to the patient.

In some embodiments, provided herein is a method for the treatment of dyskinesia due to cerebral palsy, wherein the patient is a cytochrome P450 2D6 (CYP2D6) poor metabolizer, comprising: orally administering once daily to the patient a therapeutically effective amount of the valbenazine, or an isotopic variant thereof, or a pharmaceutically acceptable salt of valbenazine or an isotopic variant thereof.

In some embodiments, provided herein is a method of administering the valbenazine, or an isotopic variant thereof, or a pharmaceutically acceptable salt of valbenazine or an isotopic variant thereof, to a patient in need thereof wherein the patient is being treated with a strong cytochrome P450 3A4 (CYP3A4) inducer, comprising: discontinuing treatment of the strong CYP3A4 inducer and then administering the valbenazine, or an isotopic variant thereof, or a pharmaceutically acceptable salt of valbenazine or an isotopic variant thereof, to the patient, thereby avoiding the use of the valbenazine, or an isotopic variant thereof, or a pharmaceutically acceptable salt of valbenazine or an isotopic variant thereof, in combination with the strong CYP3A4 inducer.

In some embodiments, provided herein is a method for the treatment of dyskinesia due to cerebral palsy in a patient in need thereof, wherein the patient is being administered a strong cytochrome P450 3A4 (CYP3A4) inducer, comprising:
discontinuing treatment of the strong CYP3A4 inducer, and then
orally administering once daily to the patient a therapeutically effective amount of the valbenazine, or an isotopic variant thereof, or a pharmaceutically acceptable salt of valbenazine or an isotopic variant thereof, thereby avoiding the concomitant use of the valbenazine, or an isotopic variant thereof, or a pharmaceutically acceptable salt of valbenazine or an isotopic variant thereof, with the strong CYP3A4 inducer.

In some embodiments, provided herein is a method for the treatment of dyskinesia due to cerebral palsy, wherein the patient is also being administered a strong cytochrome P450 3A4 (CYP3A4) inhibitor, comprising: orally administering once daily to the patient the valbenazine, or an isotopic variant thereof, or a pharmaceutically acceptable salt of valbenazine or an isotopic variant thereof, in an amount equivalent to about 40 mg as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the treatment of dyskinesia due to cerebral palsy in a patient, comprising:
(a) orally administering to the patient a therapeutically effective amount of the valbenazine, or an isotopic variant thereof, or a pharmaceutically acceptable salt of valbenazine or an isotopic variant thereof;
(b) subsequently determining that the patient is being administered a strong cytochrome P450 3A4 (CYP3A4) inhibitor; and
(c) reducing dosage of the valbenazine, or an isotopic variant thereof, or a pharmaceutically acceptable salt of valbenazine or an isotopic variant thereof, administered to the patient to an amount equivalent to about 40 mg as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester once daily.

In some embodiments, provided herein is a method for the treatment of dyskinesia due to cerebral palsy, comprising:
(a) orally administering to the patient a therapeutically effective amount of the valbenazine, or an isotopic variant thereof, or a pharmaceutically acceptable salt of valbenazine or an isotopic variant thereof, wherein the therapeutically effective amount is an amount equivalent to about 40 mg as measured by(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester once daily;
(b) subsequently determining that the patient is being administered a strong cytochrome P450 3A4 (CYP3A4) inhibitor; and
(c) administering the same therapeutically effective amount of the valbenazine, or an isotopic variant thereof, or a pharmaceutically acceptable salt of valbenazine or an isotopic variant thereof, to the patient.

In some embodiments, provided herein is a method for the treatment of dyskinesia due to cerebral palsy, wherein the patient is also being co-administered digoxin, comprising:
(a) administering to the patient a therapeutically effective amount of the valbenazine, or an isotopic variant thereof, or a pharmaceutically acceptable salt of valbenazine or an isotopic variant thereof,
(b) monitoring the digoxin concentration in the patient's blood; and
(c) reducing the dose of digoxin when the digoxin exposure in the patient's blood is increased as compared with the digoxin level in a patient who is administered digoxin alone.

In some embodiments, provided herein is a method for the treatment of dyskinesia due to cerebral palsy, wherein the patient is also in need of treatment with digoxin, the method comprising:
orally administering to the patient a therapeutically effective amount of the valbenazine, or an isotopic variant thereof, or a pharmaceutically acceptable salt of valbenazine or an isotopic variant thereof, and
administering the digoxin to the patient at a reduced dose to compensate for the expected increase in exposure resulting from co-administration of the digoxin and the valbenazine, or an isotopic variant thereof, or a pharmaceutically acceptable salt of valbenazine or an isotopic variant thereof,
wherein the reduced dose is relative to what the patient would be administered if the patient is not being administered the valbenazine, or an isotopic variant thereof, or a pharmaceutically acceptable salt of valbenazine or an isotopic variant thereof.

In some embodiments, provided are methods of treating dyskinesia associated with cerebral palsy in a patient in need thereof, comprising administering to the patient a therapeutically effective amount of vesicular monoamine transport 2 (VMAT2) inhibitor according to the instant disclosure.

In some embodiments, provided are methods of treating dyskinetic movements associated with cerebral palsy in a patient in need thereof, comprising administering to the patient a therapeutically effective amount of vesicular monoamine transport 2 (VMAT2) inhibitor according to the instant disclosure.

In some embodiments, provided are methods of treating chorea associated with cerebral palsy in a patient in need thereof, comprising administering to the patient a therapeutically effective amount of vesicular monoamine transport 2 (VMAT2) inhibitor according to the instant disclosure.

In some embodiments, provided are methods of treating dystonia associated with cerebral palsy in a patient in need thereof, comprising administering to the patient a therapeutically effective amount of vesicular monoamine transport 2 (VMAT2) inhibitor according to the instant disclosure.

In some embodiments, provided are methods of treating chorea and dystonia associated with cerebral palsy in a patient in need thereof, comprising administering to the patient a therapeutically effective amount of vesicular monoamine transport 2 (VMAT2) inhibitor according to the instant disclosure.

In some embodiments, provided are methods of treating choreoathetosis associated with cerebral palsy in a patient in need thereof, comprising administering to the patient a therapeutically effective amount of vesicular monoamine transport 2 (VMAT2) inhibitor according to the instant disclosure.

In some embodiments, provided are methods of treating choreoathetosis and dystonia associated with cerebral palsy in a patient in need thereof, comprising administering to the patient a therapeutically effective amount of vesicular monoamine transport 2 (VMAT2) inhibitor according to the instant disclosure.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1: Phase 3 Trial

A Phase 3, randomized, double blind, placebo-controlled parallel-design study to evaluate the efficacy, safety, and tolerability of valbenazine in subjects with dyskinesia due to cerebral palsy will be conducted. The study will consist of a 6-week screening period, a 14-week double-blind treatment period, a 2-week washout, a 32-week open-label treatment period, and a 2-week washout followed by a follow-up visit. The overall duration of the study is approximately 56 weeks, including a double-blind placebo-controlled treatment period followed by an open-label valbenazine treatment period. Subjects with a medically confirmed diagnosis of CP of the dyskinetic type (i.e., the predominant movement disorder is dystonia and/or choreoathetosis and is non-progressive) will be eligible.

Eligible subjects will be randomized 1:1 into one of 2 groups (valbenazine or placebo) stratified by age (6 to 11 years, 12 to 17 years, or 18 to 70 years) and stable concomitant botulinum toxin regimen (yes or no) at baseline (Day 1). Subjects who are on an established botulinum toxin regimen (ie, stable doses and injection schedule for at least 6 months prior to screening) and plan to have a stable injection schedule through the end of Week 16 will be considered to have a stable concomitant botulinum toxin regimen for randomization.

The double-blind treatment period will consist of a titration phase (6 weeks) and a maintenance phase (8 weeks). The starting dose of valbenazine will be 20 mg for pediatric subjects weighing <50 kg and 40 mg for pediatric subjects weighing ≥50 kg and adults as shown in the table below.

The first dose of randomized study treatment (20 mg for pediatric subjects <50 kg, 40 mg for pediatric subjects ≥50 kg and adults, or placebo) will be administered at the study site under the supervision of site staff on Day 1. If 20 mg/40 mg is tolerated, the dose of study treatment will be increased to 40 mg/60 mg at the end of Week 2. If 40 mg/60 mg is tolerated, the dose of study treatment will be increased to 60 mg/80 mg at the end of Week 4. If a subject does not tolerate a dose of 40 mg (pediatric subjects <50 kg) or 60 mg (pediatric subjects ≥50 kg and adults) or higher, the investigator may decrease the subject's dose one time by 1 dose level from the highest titrated dose. A subject whose dose was decreased may have their dose re-escalated 1 dose level during the titration phase after 1 week on the lower dose, if the investigator judges that the increase would be reasonably tolerated. Dose increases may occur up to the end of Week 6. Doses will be adjusted in a blinded manner; subjects receiving placebo will undergo the dose-adjustment process.

In the maintenance phase, subjects will continue the highest individually tolerated dose received in the titration phase. The maximum daily dose during the 8-week maintenance phase is 60 mg for pediatric subjects <50 kg and 80 mg for pediatric subjects ≥50 kg and adults. If the subject cannot tolerate their highest titrated dose and did not have a dose reduction during titration, the investigator may reduce the subject's dose one time by 1 dose level through the end of Week 10 (unless the subject is receiving 20 mg [pediatric subjects <50 kg] or 40 mg [pediatric subjects ≥50 kg and adults], then treatment will be discontinued).

During the titration and maintenance phases, the investigator may assess that a dose level is not tolerated and requires dose reduction if a subject experiences an adverse event (AE) that is (1) deemed associated with the study drug, and (2) of either moderate or severe intensity, or a serious AE. Subjects who could not tolerate study treatment or discontinued treatment for any reason during the double-blind treatment period are to remain in the study to complete Week 14 study assessments unless consent is withdrawn

All study visits during the double-blind treatment period have a window of ±3 days. Following the double-blind treatment period, all subjects will have 2 weeks of washout (no treatment).

During the open-label treatment period, all subjects will receive valbenazine and will retitrate to their highest individually tolerated dose. Dose titration and maintenance will occur in the same fashion as during the double-blind period. If a subject does not tolerate a dose of 40 mg (pediatric subjects <50 kg) or 60 mg (pediatric subjects ≥50 kg and adults) or higher, the investigator may decrease the subject's dose one time by 1 dose level from the highest titrated dose during the open-label treatment period. A subject whose dose was decreased during the titration phase may have their dose re-escalated 1 dose level after 1 week on the lower dose, if the investigator judges that the increase would be reasonably tolerated. Dose increases may occur through the end Week 22 (titration phase).

Some visits during the maintenance phase of the open-label treatment period may be conducted remotely. The last dose of study treatment and final efficacy and PK assessments for the open-label treatment period will occur at the end of Week 48. Follow-up assessments will be conducted at the end of Week 50 (2 weeks after the last dose of study treatment). Study visits during the open-label treatment period have a window of +3 days during titration and +6 days during maintenance.

TABLE 1
Study Treatment Dose by Weight, Phase, and Period

| | Titration Phase | | | Maintenance Phase |
|---|---|---|---|---|
| Double-Blind Period | Weeks 1-2 | Weeks 3-4 | Weeks 5-6 | Weeks 7-14 |
| VBZ (pediatric subjects < 50 kg) | 20 mg | 40 mg | 60 mg | Highest tolerated |
| | | | | dose |
| VBZ (pediatric subjects ≥ 50 kg | 40 mg | 60 mg | 80 mg | Highest tolerated |
| and adults) | | | | dose |
| Placebo (all) | Placebo | Placebo | Placebo | Placebo |
| Open-Label Period | Weeks 17-18 | Weeks 19-20 | Weeks 21-22 | Weeks 23-48 |
| VBZ (pediatric subjects < 50 kg) | 20 mg | 40 mg | 60 mg | Highest tolerated |
| | | | | dose |
| VBZ (pediatric subjects ≥ 50 kg | 40 mg | 60 mg | 80 mg | Highest tolerated |
| and adults) | | | | dose |

VBZ, valbenazine.

Subjects must meet all of the following inclusion criteria:

1. Medically confirmed diagnosis of cerebral palsy (CP) of the dyskinetic type (ie, the predominant movement disorder is dystonia and/or choreoathetosis and is non-progressive). The dyskinetic movements must cause disability per investigator assessment, and be of at least moderate severity, as confirmed by an independent review of a video recorded standardization motor examination conducted at screening.

2. Subjects with stable medical conditions requiring medications that are not prohibited per protocol must be on stable doses of these medications for a minimum of 30 days before baseline (Day 1), and the medication regimen is expected to remain stable throughout the study. Treatments for dyskinesia or spasticity, including medications, infused medications, physical medicine modalities, or electrical stimulators, must also be at stable levels for 2 weeks prior to Day 1 and expected to remain stable throughout the end of Week 16.

In some embodiments, pediatric subjects will have a body weight (in kg) greater than or equal to the 5th percentile, but less than the 95th percentile of his/her age- and gender-matched weight percentile at screening. In some embodiments, adults will have a BMI of 15 to 47 kg/m² (inclusive) at screening (BMI is defined as the subject's weight in kilograms divided by the square of the subject's height in meters).

Subjects will be excluded from the study if they meet any of the following criteria:

1. Have a clinical diagnosis or history of dyskinesia due to condition other than CP (eg, neurodegenerative disease, drug-induced dyskinesia, genetic, stroke or brain injury past perinatal period, etc.).

2. Have inability to swallow soft solids, or requirement to take medications by gastro-enteral tube.

3. Have any suicidal behavior or suicidal ideation in the year prior to screening or on Day 1.

4. Is a substance abuser of any compound.

5. Known history of long QT syndrome or cardiac tachyarrhythmia, or clinically significant ECG abnormalities.

Subjects may be excluded from the study if they meet any of the following criteria:

1. Predominant movement disorder other than dystonia and/or choreoathetosis (ie, spasticity, ataxia, parkinsonism).

2. Gross Motor Function Classification System (GMFCS) Level V.

3. Significant musculoskeletal deformity (eg, scoliosis, contractures), spasticity, or dystonia (eg, causing minimal ability to move, rigid or fixed joint positions) that would interfere with study assessments.

4. Have clinically manifest dysphagia as defined by a Swallowing Disturbance Questionnaire (SDQ) score ≥11, inability to swallow soft solids, or requirement to take medications by gastro-enteral tube.

5. An unstable medical condition or chronic disease (including history of other neurological [including cognitive impairment, myasthenia gravis], hepatic, renal, cardiovascular, gastrointestinal, pulmonary, autoimmune, infectious or endocrine disease) that may affect study participation or results, malignancy, or medically significant illness within 30 days before baseline (Day 1).

6. Have epilepsy that is not well-controlled. Well-controlled epilepsy requires that the subject have a stable seizure frequency (<1 seizure/month), stable seriousness and semiology (based on investigator opinion) for the past 12 months, and stable use of anti-epilepsy drugs (AED) for 3 months prior to baseline (Day 1). History of major surgical procedure (eg, musculoskeletal surgery, neurosurgery) or is anticipated to undergo major surgery during the course of the study, that according to the investigator may interfere with ability to perform or comply with study procedures or requirements.

7. Have an untreated, undertreated or unstable psychiatric illness, such as depression. Subjects receiving antidepressant therapy must be on a stable dose for at least 8 weeks prior to baseline (Day 1). Monoamine oxidase inhibitors (MAOIs) are prohibited within 30 days prior to baseline (Day 1).

8. Have a score ≥11 on the depression subscale of the Hospital Anxiety and Depression Scale (HADS) at screening or Day 1 (baseline) or total T-score of >70% on the Children's Depression Inventory 2^{nd} Edition (CDI-2) Self-Report Short version.

9. Have a significant risk of suicidal behavior. Subjects with any suicidal behavior or suicidal ideation of type 4 (active suicidal ideation with some intent to act, without specific plan) or type 5 (active suicidal ideation with specific plan and intent) based on the C-SSRS in the year prior to screening (using baseline/screening version) or on Day 1 (using Since Last Visit version) will be excluded.

10. Evidence of chronic renal or liver disease based on screening laboratory test abnormalities:
Serum creatinine >1.5 times the upper limit of normal (ULN)
Aspartate aminotransferase (AST) ≥2.5 times ULN
Alanine aminotransferase (ALT) ≥2.5 times ULN
Gamma-glutamyl transferase (GGT) ≥3.0×ULN
Total bilirubin >1.5 ULN unless due to a documented diagnosis of Gilbert's syndrome

11. Have any of the following laboratory abnormalities at screening:
Hemoglobin <10 g/dL
White blood cell (WBC) count <3.0×10³/mm³
Platelet count <100,000/mm³
Absolute neutrophil count <1.0×10³/mm³

12. Have a known history of long QT syndrome or cardiac tachyarrhythmia.

13. Have screening or Day 1 ECG QT interval corrected for heart rate using Fridericia's correction (QTcF) interval of >450 msec (males) or >470 msec (females), second degree atrioventricular block type 2, ECG with third degree atrioventricular block, or other clinically significant baseline ECG abnormalities as judged by the investigator.

14. Have a positive human immunodeficiency virus antibody (HIV-Ab) test result or hepatitis B surface antigen (HBsAg) test result at screening. Subjects with positive hepatitis C virus antibody (HCV-Ab) and confirmatory positive polymerase chain reaction (PCR) reflex test results at screening will be allowed to participate in the study provided that the subject is asymptomatic as assessed by the investigator and does not meet the liver function test abnormalities for ALT, AST, GGT, and total bilirubin in exclusion criterion 12.

A description of the study treatments (valbenazine and placebo capsule) is summarized in Table 2. Study treatment allocation in double-blind treatment period is provided in Table 3.

TABLE 2
Study Treatments

| Treatment Period | Valbenazine | Placebo |
|---|---|---|
| Double-Blind | | |
| Treatment administration | Subjects will consume 1 or 2 | Subjects will consume 1 or 2 |
| | capsules per day, depending | capsules per day, depending |
| | on treatment assignment, at | on treatment assignment, at |
| | approximately the same time | approximately the same time |
| | each day. | each day. |
| Unit dose strength | 20 mg/40 mg | Not applicable |
| Dose level | 20 mg/40 mg/60 mg/80 mg | Not applicable |
| Dose formulation | Oral granules in sprinkle | Oral granules in sprinkle |
| | capsules | capsules |
| Route of administration | Oral | Oral |
| Sourcing | Provided centrally by | Provided centrally by |
| | Sponsor or designee | Sponsor or designee |
| Packaging and labeling | Valbenazine will be | Matching placebo will be |
| | supplied as capsules in | provided in the same manner |
| | child-resistant blister packs. | as the active treatment. |
| Open-Label | | |
| Treatment administration | Subjects will consume 1 | — |
| | capsule per day at | |
| | approximately the same time | |
| | each day. | |
| Unit dose strength | 20 mg/40 mg/60 mg/80 mg | — |
| Dose level | 20 mg/40 mg/60 mg/80 mg | — |
| Dose formulation | Oral granules in sprinkle | — |
| | capsules | |
| Route of administration | Oral | — |
| Sourcing | Provided centrally by | — |
| | Sponsor or designee | |
| Packaging and labeling | Valbenazine will be | — |
| | supplied as capsules in | |
| | child-resistant HDPE bottles | |

HDPE = high density polyethylene;
OLE = open-label extension;
— = not applicable

TABLE 3
Study Treatment Allocation: Double-Blind Treatment Period

| | Valbenazine Capsule Strength | | |
|---|---|---|---|
| Treatment | 20 mg | 40 mg | Placebo |
| Valbenazine 20 mg | 1 | — | 1 |
| Valbenazine 40 mg | 2 | — | — |
| Valbenazine 60 mg | 1 | 1 | — |
| Valbenazine 80 mg | — | 2 | — |
| Placebo | — | — | 2 |

In the double-blind treatment period, study treatment will be supplied as oral granules for sprinkle capsules containing 20 or 40 mg of valbenazine or placebo (encapsulated granules). All subjects will receive 2 capsules (identical in appearance), that must be taken at the same time once daily as allocated in Table 3, based on assigned treatment and dose level. In the open-label treatment period, valbenazine will be supplied as oral granules for sprinkle capsules containing 20, 40, 60, or 80 mg of valbenazine (encapsulated granules). All subjects will take 1 capsule per day during titration and maintenance, based on titration level and highest individually tolerated dose. The capsules may be swallowed whole or the contents of the capsules may be sprinkled on a soft food (eg, applesauce) and entirely consumed by the subject. Study treatment will be administered (assisted by the subject's caregiver, if applicable) once daily at approximately the same time each day.

Beginning on Day 2, study treatment will be administered (assisted by the subject's caregiver, if applicable) once daily at approximately the same time each day. The subject/caregiver will be instructed to record the date and time of the last dose of study treatment administration prior to blood sample collection for PK on applicable study visit days. Adult subjects will start at the 40 mg dose in each treatment period. For pediatric subjects, weight at baseline will be used to determine the dosing regimen in the double-blind treatment period, and weight at the Week 16 visit will be used to determine the dosing regimen during the open-label treatment period (Table 1). In each of the double-blind and open-label periods, the dose of study treatment will be increased every 2 weeks, one dose level (20 mg) at a time, to a maximum dose of 60 mg for pediatric subjects <50 kg or 80 mg for pediatric subjects ≥50 kg and adults. If a subject does not tolerate a dose of 40 mg (pediatric subjects <50 kg) or 60 mg (pediatric subjects ≥50 kg and adults) or higher, the investigator may decrease the subject's dose one time.

A subject whose dose was decreased during the titration phase may have their dose re-escalated 1 dose level after 1 week on the lower dose, if the investigator judges that the increase would be reasonably tolerated. Dose increases may occur through the end of Week 6 in the double-blind period and through the end of Week 22 in the open-label treatment period. Doses will be adjusted in a blinded manner; subjects receiving placebo will undergo the dose-adjustment process.

In the maintenance phase of the double-blind treatment period, dose reductions may occur through the end of Week 10 (unless the subject is receiving 20 mg [pediatric subjects <50 kg] or 40 mg [pediatric subjects ≥50 kg and adults], then treatment will be discontinued).

The following medications are prohibited from 30 days prior to baseline (Day 1) until the final study visit (or early termination), unless otherwise stated, as described below:

Antipsychotics or other dopamine receptor blockers: antipsychotics (eg, risperidone, olanzapine, quetiapine, aripiprazole, ziprasidone, chlorpromazine, haloperidol, fluphenazine, clozapine) and dopamine receptor blockers (eg, metoclopramide, domperidone)

Strong cytochrome P450 (CYP) 3A4 inducers: eg, phenytoin, phenobarbital, rifabutin, rifampin, primidone, St. John's Wort

Dopamine receptor agonists and precursors: eg, ropinirole (agonist); carbidopa/levodopa

MAOIs: eg, isocarboxazid, phenelzine, selegiline, tranylcypromine

VMAT2 inhibitors: reserpine, tetrabenazine, deutetrabenazine

Botulinum toxin: prohibited within 4 months of Day 1 and during the study.

As needed (prn) use: As needed use of the following medications is prohibited, when administered systemically: anticholinergics, mood stabilizers, antidepressants, strong CYP3A4 inhibitors (eg, clarithromycin, diltiazem, grapefruit juice, itraconazole, ketoconazole, nefazodone), and strong CYP2D6 inhibitors (eg, bupropion, fluoxetine, paroxetine, quinidine).

Study assessments and procedures may include the following:

Genotyping-A blood sample will be collected from randomized subjects on Day 1 for the analysis of CYP2D6 status (ie, normal, intermediate, poor, or ultra-rapid metabolizers).

The Swallowing Disturbance Questionnaire (SDQ) is a 15-item instrument that assesses subjects' difficulty in swallowing food on a scale from 0 (never) to 3 (very frequently). The SDQ is a valid and reliable instrument to determine subjects' ability to swallow. See, e.g., Cohen et al. (2011) Laryngoscope. 1383-7.

United Huntington Unified Huntington Disease Rating Scale Total Motor Score (TMS) will be administered and video recorded at screening at and independently reviewed to determine subject eligibility. Subjects must have moderate or severe DCP at screening as determined by the blinded, external UHDRS reviewer. Unified Huntington's Disease Rating Scale: reliability and consistency. Huntington Study Group. Mov Disord. (1996) 11 (2): 136-42.

Clinical Global Impression of Severity (CGI-S) will be administered at screening and baseline (Day 1). Subjects with a CGI-S score of 4 (moderate) or greater at screening and baseline will be eligible to participate in the study. Busner et al. (2007) Psychiatry (Edgmont). 4 (7): 28-37.

Patient Global Impression of Severity (PGI-S) scale will be used to assess overall severity of symptoms on a 5-point scale (range: 1=none to 5=very severe). Snyder et al. (2021) J Sleep Res 30 (1): e13141.

Caregiver Global Impression of Severity (CaGI-S) scale will be used to assess the caregiver's overall impression of the severity of the subject's symptoms over the past 4 weeks (range: 0=no symptoms to 5=very severe symptoms). Rofail et al. (2016). BMC Psychiatry 16:245.

The Gross Motor Function Classification System (GMFCS) is a 5-level standardized system originally developed to classify the gross motor function of children aged 2 to 18 years with CP. A high correlation in gross motor function using the GMFCS has been demonstrated between children and adults. Paulson et al. (2017). Children (Basel). 2017; 4 (4): 30.

The following efficacy assessments may be performed.

Unified Huntington Disease Rating Scale (UHDRS) is a scale developed by the Huntington Study Group (HSG) to assess the clinical features and course of HD. The UHDRS has undergone extensive reliability and validity testing and has been used as a major outcome measure in controlled clinical trials. The full UHDRS includes the following assessments: motor, cognitive, behavioral, independence, function, and total functional capacity (TFC).

The total motor score (TMS) portion of the UHDRS is based on a standardized motor examination to measure the severity of the motor features and consists of 31 items rated from grade 0 (not affected) to grade 4 (most severely affected), resulting in a range of 0-124 points. The TMS and its chorea and dystonia domains will be used to evaluate dyskinesia movement severity in pediatric and adult subjects with DCP. The total maximal dystonia (TMD) domain consists of items 17 through 21 of the TMS and measures dystonia in 5 different body parts including the trunk and each limb independently. The maximum score is 20. The total maximal chorea (TMC) domain consists of items 22 through 28 of the TMS and measures chorea in 7 different body parts including the face, oral-buccal-lingual region, trunk, and each limb independently. The maximum score is 28.

The functional aspects of CP will be evaluated in adults using the UHDRS TFC and functional assessment checklist. The UHDRS TFC scale assesses how the investigator judges the subject's capacity to manage work, finances, daily living, domestic chores, and their care arrangements. The TFC scale focuses on assessment of the subject's capacity rather than actual performance. The functional assessment is a 25-item yes/no checklist of common daily tasks.

Unified Huntington's Disease Rating Scale Administrator (UHDRS) will be administered and scored. The UHDRS video recording files will be reviewed and scored. The central raters will score maximal chorea (0 to 4) on 7 body regions (face, buccal-oral-lingual, trunk, right upper extremities, left upper extremities, right lower extremities, and left lower extremities), and maximal dystonia (0 to 4) on 5 body regions (trunk and each limb).

Movement Disorders—Childhood Rating Scale Part I—The severity of movement disorders in subjects aged 6 to 17 years will be evaluated using the Movement Disorders Childhood Rating Scale (MD-CRS), a validated tool for assessing movement disorders during developmental age. The MD-CRS evaluates the intensity of movement disorders in different body regions at rest and during specific tasks and assess the influence of movement disorders on motor function and daily living activities. The scale is divided into 2 parts: a general assessment in Part I and a movement disorder severity assessment in Part II: only Part I will be used in this study. Part I includes 4 sections (motor function, oral/verbal function, self care, and attention/alertness) for a total of 15 items evaluated on a scale from 0 to 4.

Movement Disorders-Childhood Rating Scale Administrator (MD-CRS) will be administered.

Clinical Global Impression of Severity (CGI-S) will be used to assess overall severity of dyskinesia on a 7-point scale (range: 1=normal, not at all ill to 7=among the most extremely ill patients).

The Clinical Global Impression of Improvement (CGI-I), which is based on a 7-point scale (range: 1=very much improved to 7=very much worse), will be used to rate the overall global improvement of dyskinesia since the initiation of study treatment. This scale is a modification of a scale developed by the Psychopharmacology Research Branch of the National Institute of Mental Health to rate the subject's overall improvement in a clinical disorder and provides a global evaluation of improvement over time from the clinician's perspective.

Patient Global Impression of Improvement-Subjects will evaluate the change in their dyskinesia symptoms since initiation of study treatment dosing by choosing one of 7 responses (very much improved, much improved, minimally improved, not changed, minimally worse, much worse, and very much worse) on the Patient Global Impression of Improvement (PGI-I).

Caregiver Global Impression of Improvement-Caregivers will evaluate the subject's improvement in dyskinesia symptoms since initiation of study treatment dosing by choosing one of 7 responses (very much improved, much improved, minimally improved, not changed, minimally worse, much worse, and very much worse) on the Caregiver Global Impression of Improvement (CaGI-I).

Cerebral Palsy Quality of Life-Child (CP QOL-Child) measures the quality of life of children with CP by assessing several aspects of a child's life including physical well-being, social well-being, emotional well-being, school, access to services, and acceptance by others. Two versions of the questionnaire will be used: a primary caregiver-proxy report for children aged 6 to 12 years, and a self-report form for children aged 9 to 12 years. The primary caregiver-proxy form contains 66 items and the child self-report form contains 52 items. Service access and primary caregiver health are only included in the primary caregiver-proxy version.

Cerebral Palsy Quality of Life-Teen (CP QOL-Teen) is a survey-based holistic measure of quality of life for adolescents aged 13 to 17 years with CP and consists of adolescent self-report and primary caregiver-proxy report versions. The 72 item adolescent questionnaire includes items on global quality of life, social well-being, emotional well-being, school well-being, physical well-being, participation, communication, and pain. The primary caregiver proxy questionnaire contains an additional 17 questions regarding access to services and caregiver health.

Quality of Life in Neurological Disorders (Neuro-QoL) is a collection of psychometrically sound, clinically relevant, health-related quality of life measurement tools for adult patients with neurological conditions. The Neuro-QoL has been demonstrated to be a reliable tool for assessing patient-reported physical functioning measures in patients with HD and has been used to detect individual changes in neurological quality of life in patients with stroke, epilepsy, amyotrophic lateral sclerosis, multiple sclerosis, and Parkinson's disease. The Lower Extremity Function Short Form and the Upper Extremity Function Short Form will be administered to subjects aged 18 to 70 years. Each measure includes 8 questions about physical abilities ranging from 1 (unable to do) to 5 (without any difficulty).

Routine safety assessments will be performed and may include the following.

Columbia-Suicide Severity Rating Scale (C-SSRS) is a validated instrument to prospectively assess suicidal ideation and behavior and can be administered to children as young as 6 years. There are versions of the questionnaire designed for use at screening (Baseline/Screening version) and at baseline and visits throughout the study (Since Last Visit version). All versions of the C-SSRS include a series of screening questions related to suicidal ideation and suicidal behavior. Subject responses of “yes” to one or more screening questions will prompt additional questions that evaluate frequency and intensity of suicidal ideation and/or behavior. Subjects with any suicidal behavior or suicidal ideation of type 4 (active suicidal ideation with some intent to act, without specific plan) or type 5 (active suicidal ideation with specific plan and intent) in the year before screening based on the C-SSRS should be excluded (exclusion criterion 9).

The Hospital Anxiety and Depression Scale (HADS) is a commonly used instrument to determine the levels of anxiety and depression that a person is experiencing. The HADS is a 14-item scale; 7 of the items relate to anxiety and 7 relate to depression. Each item is answered on a 4-point (0 to 3) response category so the possible scores range from 0 to 21 for anxiety and 0 to 21 for depression. The HADS has been validated as a measure of depression and anxiety.

The Children's Depression Inventory 2^{nd} Edition (CDI-2) is a comprehensive multirater assessment of depressive symptoms in children aged 7 to 17 years, and will be used for subjects 6 to 17 years in this study. It includes a scale to evaluate emotional problems (negative mood/physical symptoms and negative self-esteem) and functional problems (interpersonal problems and ineffectiveness). The Self-Report Short version is an efficient screening measure that contains 12 items and yields a total score that is generally comparable to the one produced by the full-length (28-item) version.

The Barnes Akathisia Rating Scale (BARS) is a validated 4-item scale to assess the presence and severity of drug-induced akathisia. This scale includes both objective items (eg, observed restlessness) and subjective items (eg, subjects' awareness of restlessness and related distress), together with a global assessment of akathisia. Global assessment is made on a scale of 0 to 5 (0-absent; 1=questionable; 2=mild akathisia; 3=moderate akathisia; 4-marked akathisia; 5=severe akathisia).

The Modified Ashworth Scale (MAS) is a muscle tone assessment scale used to evaluate resistance during passive range of motion. The MAS is measured on a scale of 0 to 4 (0=no increase in muscle tone; 1=slight increase in muscle tone, with a catch and release or minimal resistance at the end of the range of motion when an affected part(s) is moved in flexion or extension; 1+=slight increase in muscle tone, manifested as a catch, followed by minimal resistance through the remainder (less than half) of the range of motion; 2=marked increase in muscle tone throughout most of the range of motion, but affected part(s) are still easily moved; 3=considerable increase in muscle tone, passive movement difficult; 4-affected part(s) rigid in flexion or extension). The score will be recorded for the tone at the elbow and wrist of each upper limb, and knee and ankle of each lower limb.

UHDRS Items for Parkinsonism-A subset of the UHDRS TMS (items rating retropulsion pull test, finger taps, pronate/supinate hands, rigidity-arms, and bradykinesia-body) will be used to assess for parkinsonism at each visit.

The primary endpoint of the change from baseline in UHDRS TMC score based on investigator assessment will be analyzed using a linear mixed-effect repeated measures model using the scores at the end of Weeks 2, 4, 6, 8, 12, and 14. The model will include the baseline TMC score as a covariate, age (6 to 11 years versus 12 to 17 years versus 18 to 70 years), treatment group (valbenazine or placebo), visit, and treatment group-by-visit interactions as fixed effects. Day 1 assessments will be used as baseline. The primary comparison will be the contrast between treatment groups at Week 14 and the valbenazine group will be compared with the placebo group using a 2-sided test with a significance level of 0.025.

The other primary endpoint of change from baseline in UHDRS TMD score will be analyzed using the same analysis method as that used for change from baseline in UHDRS TMC.

The key secondary endpoint of change in CGI-S score from baseline to Week 14 will use a similar analysis method as used for the primary endpoint. The CGI-S score collected on Day 1 will be considered the baseline measurement. The full analysis set will be used for the primary analysis.

The additional secondary efficacy endpoints are as follows:

The PGI-I score at Week 14.

The CaGI-I score at Week 14.

The CGI-I score at Week 14.

Change in the UHDRS functional assessment and functional capacity scores from baseline to Week 14 (ages 18 to 70 years only).

Change in MD-CRS Part I score from baseline to Week 14 (ages 6 to 17 years only).

Change in Quality of Life in Neurological Disorders (Neuro-QoL) (Lower Extremity Function Short Form, Upper Extremity Function Short Form, and Satisfaction with Social Roles and Activities Short Form) from baseline to Week 14.

Change in Cerebral Palsy Quality of Life-Teen (CP QOL-Teen) from baseline to Week 14.

Change in CP QOL-Teen (caregiver-proxy report) from baseline to Week 14 (ages 13 to 17 years).

Change in Cerebral Palsy Quality of Life-Child (CP QOL-Child) from baseline to Week 14 (ages 6 to 12 years).

Change in CP QOL-Child (caregiver-proxy report) from baseline to Week 14 (ages 6 to 12 years).

Change in pain assessment from baseline to Week 14 using the Faces Pain Scale-Revised (FPS-R).

Change in the UHDRS TMS from baseline to Week 14.

Change from baseline in the UHDRS functional assessment and functional capacity scores will be analyzed using subjects in the full analysis set who are 18 to 70 years old, change from baseline in MD-CRS Part 1 score will be analyzed using subjects in the full analysis set who are 6 to 17 years old, while the other secondary endpoints will use the full analysis set for the primary analysis. Age group will be included as a covariate for the endpoints for which more than one age group is included in the analysis set.

The patient- and caregiver-reported outcome secondary endpoints using Neuro-QoL and CP-QOL will be analyzed using linear mixed-effect repeated measures models using the scores at the end of Weeks 6 and 14. The models will include the baseline value as a covariate, treatment group (valbenazine or placebo), visit, and treatment group-by-visit interactions as fixed effects. Subject will be included as a random effect. Day 1 assessments will be used as baseline. Each endpoint will be analyzed in the age group for which it is applicable using the definition of the full analysis set in addition to age.

Safety data from this study will be analyzed using the safety analysis set. The subject incidence of treatment-emergent AEs will be tabulated by treatment group for AEs, SAEs, fatal AEs, and AEs leading to discontinuation of study treatment. Descriptive statistics by treatment group will be generated for additional safety data, including select laboratory analytes, vital signs, ECG parameters, C-SSRS, HADS, CDI-2, UHDRS TMS (items for parkinsonism), MAS, and BARS, which will be further described in the SAP.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of treating dyskinesia due to cerebral palsy in a patient in need thereof, comprising administering to the patient a therapeutically effective amount of valbenazine, or an isotopic variant thereof, or a pharmaceutically acceptable salt of valbenazine or an isotopic variant thereof.

2. The method of claim 1, wherein the dyskinesia due to cerebral palsy is characterized by abnormal involuntary movements of the dystonic type.

3. The method of claim 1, wherein the dyskinesia due to cerebral palsy is characterized by abnormal involuntary movements of the athetoid type.

4. The method of claim 1, wherein the dyskinesia due to cerebral palsy is characterized by abnormal involuntary movements of the chorea type.

5. The method of any one of the preceding claims, wherein the valbenazine, or an isotopic variant thereof, or a pharmaceutically acceptable salt of valbenazine or an isotopic variant thereof, is administered via a titration scheme that comprises the up-titration over a period of no more than about six weeks until an optimized dose is administered.

6. The method of claim 5, wherein the titration scheme comprises administering the valbenazine, or an isotopic variant thereof, or a pharmaceutically acceptable salt of valbenazine or an isotopic variant thereof, at an initial dose equivalent to about 20 mg of valbenazine free base once daily for about two weeks for pediatric patients having a body weight less than 50 kg and, provided that the patient tolerates the initial dose and that the patient has not achieved satisfactory control of abnormal involuntary movements, increasing the dose and administering the increased dose to the patient.

7. The method of claim 6, wherein the increased dose is equivalent to about 40 mg of valbenazine free base once daily.

8. The method of claim 6 or 7, wherein the titration scheme further comprises administering the valbenazine, or an isotopic variant thereof, or a pharmaceutically acceptable salt of valbenazine or an isotopic variant thereof, at said increased dose for about two weeks.

9. The method of claim 7 or 8, wherein if the patient does not tolerate the increased dose, the optimized dose is the initial dose.

10. The method of claim 9, wherein if the patient tolerates the increased dose and if the patient has achieved satisfactory control of abnormal involuntary movements, the optimized dose is the increased dose.

11. The method of claim 9 or 10, further comprising administering the optimized dose of the valbenazine, or an isotopic variant thereof, or a pharmaceutically acceptable salt of valbenazine or an isotopic variant thereof, to the patient.

12. The method of claim 11, wherein if the patient tolerates the increased dose and if the patient has not achieved satisfactory control of abnormal involuntary movements, the method further comprises increasing the dose.

13. The method of claim 12, wherein the further increased dose is equivalent to about 60 mg of valbenazine free base once daily.

14. The method of claim 12 or 13, wherein if the patient does not tolerate the further increased dose, the optimized dose is the increased dose.

15. The method of claim 12 or 13, wherein if the patient tolerates the further increased dose and if the patient has achieved satisfactory control of abnormal involuntary movements, the optimized dose is the further increased dose.

16. The method of claim 14 or 15, further comprising administering the optimized dose of the valbenazine, or an isotopic variant thereof, or a pharmaceutically acceptable salt of valbenazine or an isotopic variant thereof, to the patient.

17. The method of claim 5, wherein the titration scheme comprises administering the valbenazine, or an isotopic variant thereof, or a pharmaceutically acceptable salt of valbenazine or an isotopic variant thereof, at an initial dose equivalent to about 40 mg of valbenazine free base once daily for about two weeks for patients having a body weight greater than or equal to 50 kg and, provided that the patient tolerates the initial dose and that the patient has not achieved satisfactory control of abnormal involuntary movements, increasing the dose and administering the increased dose to the patient.

18. The method of claim 17, wherein the increased dose is equivalent to about 60 mg of valbenazine free base once daily.

19. The method of claim 17 or 18, wherein the titration scheme further comprises administering the valbenazine, or an isotopic variant thereof, or a pharmaceutically acceptable salt of valbenazine or an isotopic variant thereof, at said increased dose for about two weeks.

20. The method of claim 19, wherein if the patient does not tolerate the increased dose, the optimized dose is the initial dose.

21. The method of claim 19, wherein if the patient tolerates the increased dose and patient has achieved satisfactory control of abnormal involuntary movements, the optimized dose is the increased dose.

22. The method of claim 20 or 21, further comprising administering the optimized dose of the valbenazine, or an isotopic variant thereof, or a pharmaceutically acceptable salt of valbenazine or an isotopic variant thereof, to the patient.

23. The method of claim 19, wherein if the patient tolerates the increased dose and if the patient has not achieved satisfactory control of abnormal involuntary movements, the method further comprises increasing the dose.

24. The method of claim 23, wherein the further increased dose is equivalent to about 80 mg of valbenazine free base once daily.

25. The method of claim 23 or 24, wherein if the patient does not tolerate the further increased dose, the optimized dose is the increased dose.

26. The method of claim 23 or 24, wherein if the patient tolerates the increased dose and if the patient has achieved satisfactory control of abnormal involuntary movements, the optimized dose is the further increased dose.

27. The method of claim 23 or 24, further comprising administering the optimized dose of the valbenazine, or an isotopic variant thereof, or a pharmaceutically acceptable salt of valbenazine or an isotopic variant thereof, to the patient.

28. The method of any one of the preceding claims, wherein the patient is 6 to 11 years.

29. The method of claim 28, wherein the patient weight <50 kg.

30. The method of any one of claims 1 to 27, wherein the patient is 12 to 17 years.

31. The method of claim 30, wherein the patient weight <50 kg.

32. The method of claim 30, wherein the patient weight ≥50 kg.

33. The method of any one of claims 1 to 27, wherein the patient is 18 or older.

34. The method of claim 33, wherein the patient weight ≥50 kg.

35. The method of any one of the preceding claims, wherein prior to administration, the patient has moderate or severe dyskinesia due to cerebral palsy.

36. The method of any one of the preceding claims, wherein prior to administration, the patient has a Clinical Global Impression of Severity (CGI-S) score of at least 4.

37. The method of any one of the preceding claims, wherein the treatment results in a change in the UHDRS TMC score, as assessed by an investigator.

38. The method of any one of the preceding claims, wherein the treatment results in a change in the UHDRS TMD score.

39. The method of any one of the preceding claims, wherein the treatment results in a change in CGI-S score.

40. The method of any one of the preceding claims, wherein the treatment results in a change in the UHDRS functional assessment and functional capacity scores.

41. The method of any one of the preceding claims, wherein the treatment results in a change in MD-CRS Part I score.

42. The method of any one of the preceding claims, wherein the treatment results in a change in Neuro-QoL.

43. The method of any one of the preceding claims, wherein the treatment results in a change in CP QOL-Teen.

44. The method of any one of the preceding claims, wherein the treatment results in a change in CP QOL-Teen (caregiver-proxy report).

45. The method of any one of the preceding claims, wherein the treatment results in a change in CP QOL-Child.

46. The method of any one of the preceding claims, wherein the treatment results in a change in CP QOL-Child (caregiver-proxy report).

47. The method of any one of the preceding claims, wherein the treatment results in a change in the UHDRS TM.

48. The method of any one of the preceding claims, wherein valbenazine, or a pharmaceutically acceptable salt thereof, is administered.

49. The method of any one of the preceding claims, wherein a pharmaceutically acceptable salt of valbenazine is administered.

50. The method of claim 49, wherein the pharmaceutically acceptable salt is a tosylate salt.

51. The method of claim 50, wherein the tosylate salt is a ditosylate salt.

52. The method of claim 51, wherein the ditosylate salt is polymorphic Form I.
